(19) United States
(12) Patent Application Publication
(10) Pub. No.: US 2006/0025477 A1
(43) Pub. Date: Feb. 2, 2006

(54) Analogs of isovaleramide, a pharmaceutical composition including the same, and a method of treating central nervous system conditions or diseases

(76) Inventors: Linda D. Artman, Salt Lake City, UT (US); Manuel F. Balandrin, Salt Lake City, UT (US); Scott T. Moe, Marlborough, MA (US); Bradford C. Van Wagenen, Salt Lake City, UT (US); Amir Pesyan, Salt Lake City, UT (US)

Correspondence Address: TRASK BRITT, P.O. BOX 2550, SALT LAKE CITY, UT, 84110, US

(21) Appl. No.: 11/188,214

(22) Filed: Jul. 22, 2005

Related U.S. Application Data
(60) Provisional application No. 60/590,373, filed on Jul. 22, 2004.

(51) Int. Cl. A61K 31/19; A61K 31/185; A61K 31/16

(52) U.S. Cl. 514/553; 514/557; 514/613; 514/625

(57) ABSTRACT

An isovaleramide analog having at least one of an increased potency, an increased half-life, and an increased stability compared to isovaleramide. The isovaleramide analog is a cyclic analog or a noncyclic analog. The isovaleramide analog is formulated into a pharmaceutical composition. A method of treating a central nervous system condition or disease is also disclosed. The method comprises administering an isovaleramide analog to a patient suffering from the central nervous system condition or disease.

### CROSS-REFERENCE TO RELATED APPLICATIONS

This application claims the benefit of U.S. Provisional Patent Application Ser. No. 60/590,373, filed Jul. 22, 2004, for ANALOGS OF ISOVALERAMIDE, A PHARMACEUTICAL COMPOSITION INCLUDING THE SAME, AND A METHOD OF TREATING CENTRAL NERVOUS SYSTEM CONDITIONS OR DISEASES.

### FIELD OF THE INVENTION

The present invention relates to analogs of isovaleramide. More specifically, the present invention relates to isovaleramide analogs that exhibit increased stability and half-life, while producing similar or increased biologic activity.

### BACKGROUND OF THE INVENTION

A number of pathological conditions (e.g., epilepsy, stroke, bipolar affective disorder, migraine, anxiety, spasticity, spinal cord injury, and chronic neurodegenerative disorder), and diseases (e.g., Parkinson's disease, Huntington's disease, and Alzheimer's disease) are characterized by abnormalities in the normal function of the central nervous system (“CNS”). These conditions and diseases typically respond to pharmacologic intervention with compounds or substances that modulate CNS activity. Compounds with this activity include isovaleramide and isovaleric acid, which have been disclosed to treat abnormalities of the CNS, such as epilepsy.

While isovaleramide has good CNS activity, orally administered isovaleramide has a short half-life in humans. Orally administered isovaleramide is readily absorbed from the gastrointestinal tract and has a half-life of about 2.5 hours for doses ranging from 100 mg to 1600 mg. The short half-life may require frequent administration to sustain a therapeutic concentration of the isovaleramide without adverse effects, and where frequent dosing schedules are required, the cost of therapy may increase. In addition, as the required dosing frequency increases, patient compliance tends to decrease.

It would be desirable to provide additional compounds that modulate CNS activity and have an increased half-life, a similar or increased activity, and/or an increased stability compared to that of isovaleramide.

### BRIEF SUMMARY OF THE INVENTION

The present invention relates to isovaleramide analogs that include compounds with similar or increased potency, increased half-life, and/or increased stability compared to isovaleramide. A compound of the present invention may be a cyclic or a noncyclic analog of isovaleramide and may be selected from the group consisting of 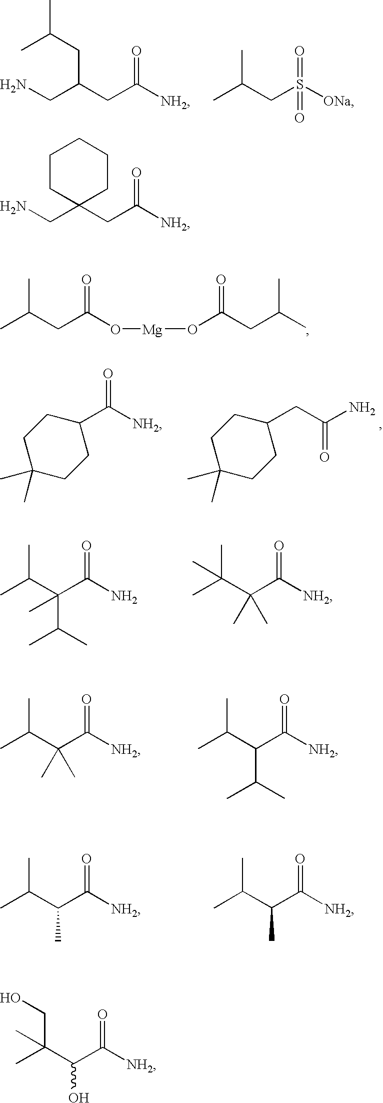
and mixtures thereof.

The present invention also relates to a pharmaceutical composition that includes an isovaleramide analog and a pharmaceutically acceptable carrier. The isovaleramide analog included in a pharmaceutical composition of the present invention is selected from the group consisting of 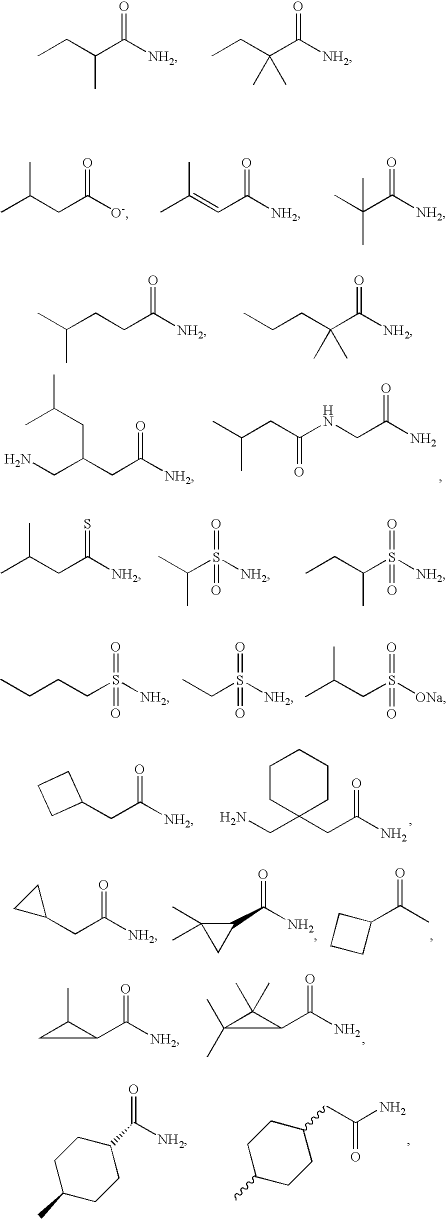 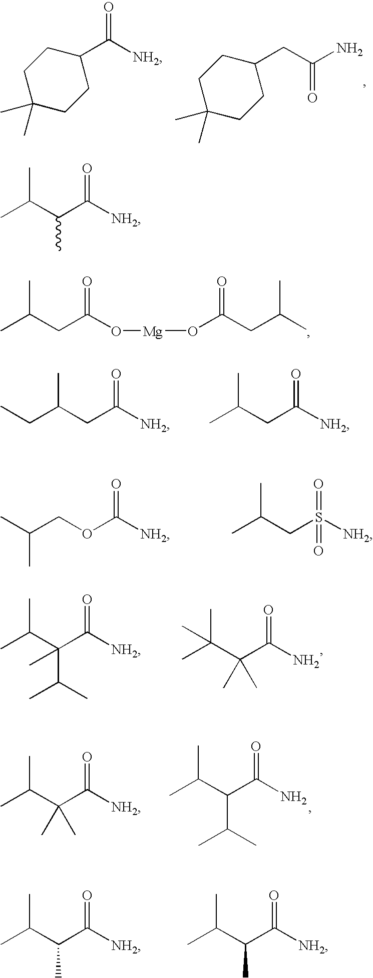
and mixtures thereof.

The pharmaceutically acceptable carrier included in a pharmaceutical composition of the present invention may be any suitable carrier. For example, the pharmaceutically acceptable carrier may include a carrier selected from the group consisting of calcium carbonate, calcium phosphate, calcium sulfate, sucrose, dextrose, lactose, fructose, xylitol, sorbitol, starch, starch paste, cellulose derivatives, gelatin, polyvinylpyrrolidone, sodium chloride, dextrins, stearic acid, magnesium stearate, calcium stearate, vegetable oils, polyethylene glycol, sterile phosphate-buffered saline, saline, Ringer's solutions, and mixtures thereof. A pharmaceutical composition of the present invention includes an amount of an isovaleramide analog sufficient to allow therapeutically effective dosing of the isovaleramide analog from a desired dosage form. In one embodiment, the isovaleramide analog may be present in an amount of from approximately 1% by weight to approximately 95% by weight of a total weight of the pharmaceutical composition. The isovaleramide analog may be present in the pharmaceutical composition in a range of from approximately 10 mg to approximately 1200 mg.

The present invention also relates to a method of treating a central nervous system condition or disease. The method includes administering an isovaleramide analog to a patient suffering from a central nervous system condition or disease. For the sake of example only, the central nervous system condition or disease may include convulsions, spasticity, affective mood disorders, neuropathic pain syndromes, neurodegenerative disorders, headaches, premenstrual syndrome, menstrual discomfort, hyperexcitability in children, restlessness syndromes, movement disorders, cerebral trauma, anxiety-related disorders, or symptoms of substance abuse/craving. The isovaleramide analog may be one of the previously described cyclic or noncyclic analogs of isovaleramide.

An isovaleramide analog of the present invention may be administered by any appropriate method. For example, an isovaleramide analog according to the present invention may be administered orally, transversally, transmucosally, intravenously, intraperitoneally, subcutaneously, rectally, nasally, bucally, or intramuscularly. Where an isovaleramide analog of the present invention is administered in an oral dosage form, the dosage form is typically selected from tablets, such uncoated and coated tablets, caplets, gelcaps, and capsules. Alternatively, an isovaleramide analog of the present invention may be orally administered using a liquid dosage form such as a solution, a suspension, a syrup, or an elixir. A therapeutically effective amount of the compound (such as, e.g., from approximately 10 mg to approximately 1200 mg) may be administered to the patient. A pharmaceutical composition of the present invention can be formulated to allow administration of the pharmaceutical composition by any appropriate method.

### BRIEF DESCRIPTION OF THE SEVERAL VIEWS OF THE DRAWINGS

While the specification concludes with claims particularly pointing out and distinctly claiming that which is regarded as the present invention, the advantages of this invention may be more readily ascertained from the following description of the invention when read in conjunction with

FIG. 1, which shows chemical structures of isovaleramide and of isovaleramide analogs that exemplify embodiments of the analogs included in the present invention.

### DETAILED DESCRIPTION OF THE INVENTION

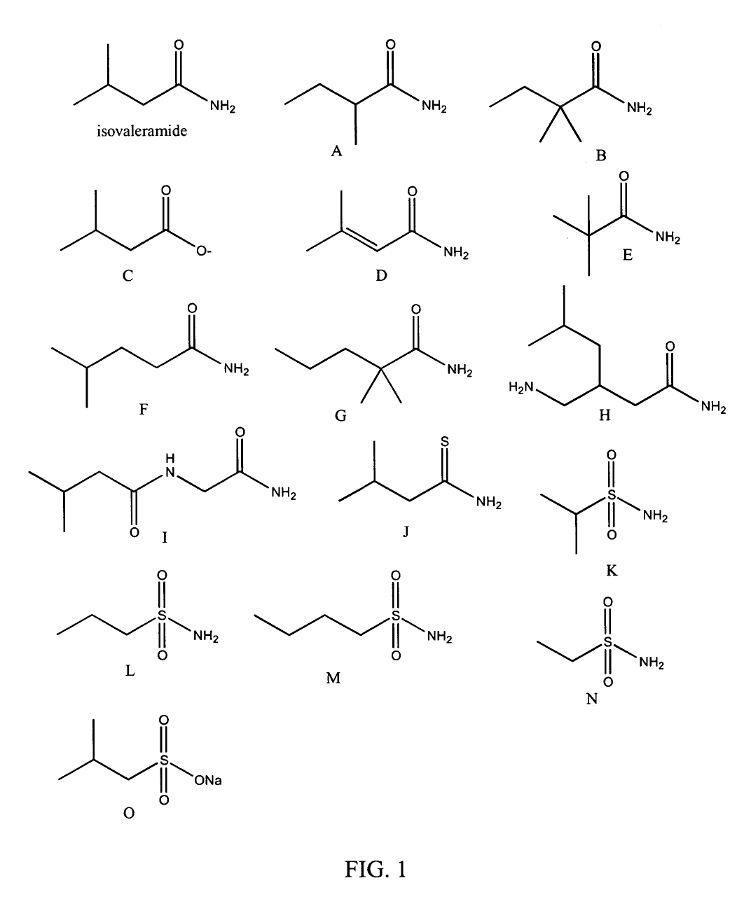

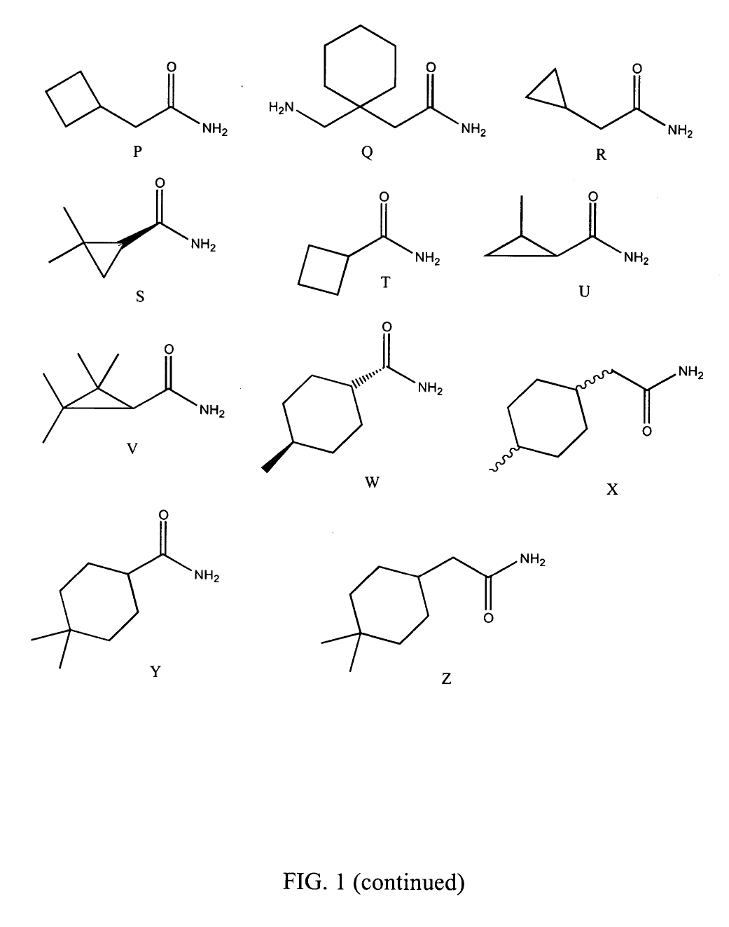

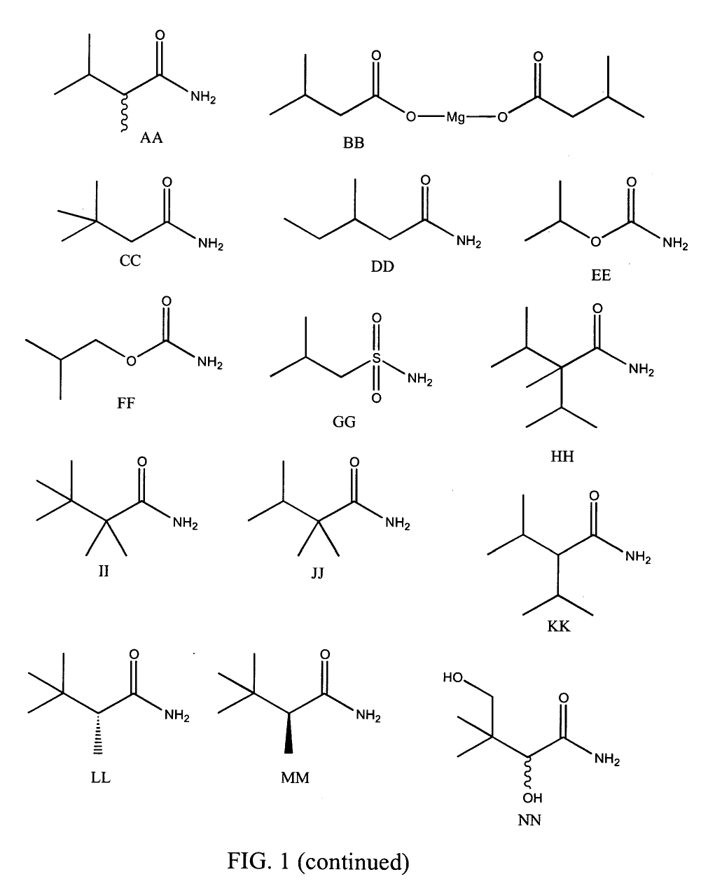

The present invention includes noncyclic and cyclic analogs of isovaleramide. An isovaleramide analog of the present invention may be an amide, sulfonamide, carboxylic acid salt, thioamide, or sulfonic acid salt analog of isovaleramide. Chemical structures of isovaleramide and of isovaleramide analogs of the present invention are shown in FIG. 1. Noncyclic isovaleramide analogs according to the present invention include, but are not limited to, one of structures A-O or AA-NN shown in FIG. 1. Cyclic isovaleramide analogs according to the present invention include, but are not limited to, one of structures P-Z shown in FIG. 1. An isovaleramide analog of the present invention may also be a derivative or structural isomer of one of the structures shown in FIG. 1. Isovaleramide analogs of the present invention may have one or more similar or increased effects on CNS activity compared to the activity of isovaleramide. As such, isovaleramide analogs of the present invention may be at least as potent as isovaleramide. The isovaleramide analogs of the present invention may also be more chemically and/or metabolically stable and/or have an increased half-life compared to isovaleramide.

Isovaleramide analogs of the present invention may either be obtained commercially or may be prepared by synthetic methods known in the art. For instance, Compound I, Compound S ((S)-(+)-2,2-dimethylcyclopropanecarboxamide), and Compound NN are commercially available from, for example, Sigma-Aldrich Chemical Co. (Milwaukee, Wis.) or BRI.

To synthesize an amide analog of the present invention, a carboxylic acid precursor of the analog may be reacted with thionyl chloride or oxalyl chloride to form an acid chloride intermediate, as shown in the following reaction scheme: 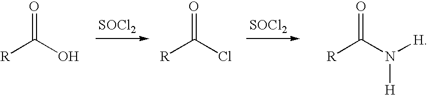
The acid chloride intermediate may be reacted with excess ammonia or an amine to form the amide analog. Carboxylic acid precursors of many isovaleramide analogs are commercially available, such as from Sigma-Aldrich Chemical Co., Acros Organics B.V.B.A. (Geel, Belgium), which is a company related to Fischer Scientific International Inc., Pfaltz & Bauer (Waterbury, Conn.), and Fluka (Buchs, Switzerland). The carboxylic acid precursor may be heated (typically at reflux) in an excess of thionyl chloride to generate the acid chloride intermediate. Alternatively, the acid chloride intermediate may be generated by treating a solution of the carboxylic acid precursor in dichloromethane at ambient temperature with an approximately 10% excess of oxalyl chloride and a catalytic amount of dimethylformamide (“DMF”). After the acid chloride intermediate is formed, excess reagent and solvents may be removed. The acid chloride intermediate may then be dissolved, such as in dichloromethane, and transferred to a cooled solution of excess ammonia in diethyl ether or dichloromethane. The solution may include greater than approximately 2 molar equivalents of ammonia. Completion of the reaction may be followed by removing excess reagents or starting materials. For example, the reaction mixture may be diluted with diethyl ether, followed by extraction with dilute acid (e.g., 1 NHCl) to remove excess ammonia. A dilute base (e.g., 1 N NaOH) may be used to remove unreacted carboxylic acid precursor. Further purification of the amide analog may be achieved by methods known in the art, such as by recrystallization, distillation, or chromatography.

If a carboxylic acid precursor is not commercially available, the carboxylic acid precursor may be synthesized by techniques known in the art. For example, a carboxylic acid ester may be deprotonated with a strong normucleophilic base, such as lithium diisopropylamide (“LDA”), followed by alkylation with methyl iodide or methyl trifluoromethanesulfonate to form the carboxylic acid precursor. The alkylated carboxylic acid ester may then be hydrolyzed and converted to the corresponding amide by the previously described methods.

If the isovaleramide analog includes one or more asymmetric centers, individual enantiomers may be prepared from optically active starting materials. If the enantiomers are present as a mixture, the individual enantiomers may be separated from one another by traditional methods of resolution, such as by fractional crystallization of salts with chiral amines or by preparation of amides with chiral amides, chromatographic separation, and hydrolysis of the amides. Alternatively, the isovaleramide analog may be prepared by well known methods of asymmetric synthesis, such as by alkylation of an ester or amide of the acid prepared using a chiral auxiliary.

To synthesize a sulfonamide analog of isovaleramide, gaseous ammonia may be reacted with a sulfonyl chloride precursor. Sulfonyl chloride precursors of many of the isovaleramide analogs are commercially available. To synthesize a carboxylic acid salt analog of isovaleramide, magnesium hydroxide (“Mg(OH)₂”), for example, may be reacted with isovaleric acid, which is available from Lancaster Synthesis (Windham, N.H.). To synthesize a sulfonic acid salt analog of isovaleramide, sodium sulfite (“Na₂SO₃”), for example, may be reacted with a halogenated alkyl precursor.

The chemical structure of each of the isovaleramide analogs is characterized by ¹H nuclear magnetic resonance (“NMR”) and/or gas chromatography-coupled mass spectrometry (“GC/MS”), as known in the art.

Isovaleramide analogs of the present invention may be active at a receptor that modulates CNS activity, such as by inhibiting or activating the receptor, and isovaleramide analogs of the present invention may modulate CNS activity by enhancing inhibitory neurotransmissions centrally, or decreasing excitatory neurotransmissions centrally. The isovaleramide analogs of the present invention may modulate the CNS activity without producing excessive sedation, muscle weakness, fatigue, teratogenicity, or hepatotoxicity in a patient to whom the isovaleramide analog is administered. As such, the isovaleramide analogs of the present invention may be effective in treating one or more CNS conditions or diseases, such as convulsions, spasticity, affective mood disorders, neuropathic pain syndromes, neurodegenerative disorders, headaches, premenstrual syndrome, menstrual discomfort, hyperexcitability in children, restlessness syndromes, movement disorders, cerebral trauma, anxiety-related disorders, and symptoms of substance abuse/craving, such as the symptoms of smoking cessation or treatment of alcoholism.

Convulsive conditions or diseases may include epilepsy, simple partial seizures, complex partial seizures, secondarily generalized seizures, status epilepticus, and trauma-induced seizures, such as those following head injury or surgery. Conditions or diseases associated with spasticity may include multiple sclerosis, cerebral palsy, stroke, trauma or injury to the spinal cord, and closed head trauma. Conditions or diseases associated with affective mood disorder may include, but are not limited to, depression, dysphoric mania, bipolar mood disorder, mania, schizoaffective disorder, traumatic brain injury-induced aggression, post-traumatic stress disorder, panic states, and behavioral dyscontrol syndromes. Conditions or diseases associated with neuropathic pain syndromes include, but are not limited to, stroke, trauma, multiple sclerosis, cancer, and diabetes. Conditions or diseases associated with headaches include, but are not limited to, chronic headaches, cluster headaches, and migraine headaches. Conditions or diseases associated with restlessness syndromes include, but are not limited to, drug-induced restlessness (tardive, chronic, and withdrawal akathisias), such as drug-induced extrapyramidal symptoms, restless limb syndromes (restless leg syndrome), and sleep-related periodic leg movements. Conditions or diseases associated with movement disorders include, but are not limited to, Parkinson's disease, Huntington's chorea, tardive dyskinesia, dystonias, and stiff-man syndrome. Conditions or diseases associated with neurodegeneration include cerebral insults, such as ischemia, trauma, seizure, or hypoglycemia. Symptoms associated with anxiety-related disorders include, but are not limited to, restlessness, nervousness, inability to concentrate, tension, overaggressiveness, irritability, and insomnia.

The CNS condition or disease is treated by administering to a patient in need of treatment a pharmaceutical composition that includes at least one isovaleramide analog according to the present invention. The pharmaceutical composition includes an isovaleramide analog, or a mixture of such analogs, in combination with a pharmaceutically acceptable carrier. If the isovaleramide analog is a chiral compound, the pharmaceutical composition may include one of the enantiomers of the isovaleramide analog or may include a racemic mixture of the enantiomers. The isovaleramide analog and the pharmaceutically acceptable carrier may be combined in amounts that produce a pharmaceutical composition that allows dosing of the isovaleramide analog in a therapeutically effective amount. For example, the isovaleramide analog may constitute from approximately 1% by weight to approximately 95% by weight of a total weight of the pharmaceutical composition. In one embodiment, the isovaleramide analog constitutes from approximately 10% by weight to approximately 85% by weight of the total weight of the pharmaceutical composition. In another embodiment, the isovaleramide analog constitutes from approximately 20% by weight to approximately 75% by weight of the total weight of the pharmaceutical composition.

The isovaleramide analog(s) included in a pharmaceutical composition of the present invention may be present in the pharmaceutical composition as a salt, such as a pharmaceutically acceptable salt. Examples of pharmaceutically acceptable salts include, but are not limited to, acetate, alkylamine, aluminum, ammonium, benzathine, benzenesulfonate, besylate, benzoate, bicarbonate, bitartrate, calcium, calcium edetate, camsylate, carbonate, citrate, chloride, chloroprocaine, choline, cyclohexylsulfamate, diethanolamine, edetate, edisylate, estolate, esylate, ethanesulfonatefumarate, ethylenediamine, gluceptate, gluconate, glutamate, glycolylarsanilate, hexylresorcinate, hydrabamine, hydrobromide, hydrochloride, hydroxynaphthoate, isethionate, lactate, lactobionate, lithium, magnesium, malate, maleate, mandelate, meglumine, mesylate, methanesulfonate, mucate, napsylate, nitrate, pamoate (embonate), pantothenate, phosphate/disphosphate, polygalacturonate, potassium, procaine, quinate, salicylate, sodium, stearate, subacetate, succinate, sulfate, sulfamate, tannate, tartrate, teoclate, p-toluenesulfonate, and zinc salts of the isovaleramide analog. Other pharmaceutically acceptable salts are known in the art and may also be used.

The pharmaceutically acceptable carrier includes a suitable excipient and/or auxiliary whose administration is tolerated by the patient. Pharmaceutically acceptable carriers are known in the art and include, but are not limited to, calcium carbonate, calcium phosphate, calcium sulfate, sucrose, dextrose, lactose, fructose, xylitol, sorbitol, starch, starch paste, cellulose derivatives, gelatin, polyvinylpyrrolidone, sodium chloride, dextrins, stearic acid, magnesium stearate, calcium stearate, vegetable oils, polyethylene glycol, sterile phosphate-buffered saline, saline, Ringer's solutions, and mixtures thereof.

The pharmaceutical composition is formulated as known in the art. For instance, the isovaleramide analog, or the mixture of isovaleramide analogs, may be combined with the pharmaceutically acceptable carrier and processed into a desired dosage form. The pharmaceutical composition may be produced by mixing, dissolving, granulating, dragee-making, levigating, emulsifying, encapsulating, entrapping, or lyophilizing the isovaleramide analog(s) with the pharmaceutically acceptable carrier.

The patient in need of treatment may be a human patient suffering from the CNS condition or disease and may exhibit one or more clinically recognized symptoms of the CNS condition or disease. A patient diagnosed with one of the above-mentioned CNS conditions or diseases exhibits at least one symptom that is alleviated by modulating CNS activity. Administering an isovaleramide analog according to the present invention to the patient reduces or eliminates at least one symptom of the CNS condition or disease. An isovaleramide analog according to the present invention may also have increased chemical and/or metabolic stability and an increased half-life compared to that of isovaleramide. In one embodiment, administering the isovaleramide analog to the patient reduces or eliminates at least one symptom associated with seizures. The pharmaceutical composition may also be used to treat similar conditions or diseases in other primates, domestic herd animals (cows, sheep, etc.), or pets (horses, dogs, cats, etc.).

A pharmaceutical composition (and, therefore, an isovaleramide analog) is administered to a patient in a manner that provides a therapeutically effective amount of the one or more isovaleramide analogs included in the pharmaceutical composition. As used herein, the phrase “therapeutically effective amount” refers to an amount of the isovaleramide analog(s) that results in a detectable change in the CNS activity or one or more symptoms suffered by the patient who receives the pharmaceutical composition. The amount or dose of isovaleramide analog included in or dosed from the pharmaceutical composition may be based on the potency and the half-life of the isovaleramide analog. The patient's age, weight, height, sex, general medical condition, and previous medical history may also affect the amount of isovaleramide analog to be included in or dosed from the pharmaceutical composition.

The pharmaceutical composition may be administered orally using a solid oral dosage form, such as an enteric-coated tablet, a caplet, a gelcap, or a capsule. Alternatively, suitable oral dosage forms for a pharmaceutical composition of the present invention also include liquid oral dosage forms, such as solutions, suspensions, syrups or elixirs. Liquid formulations that provide a suitable dose of the isovaleramide analog in 1 or 2 teaspoonfuls may be employed for convenient administration. The dosage of the isovaleramide analog used to reduce or eliminate the patient's symptoms may range from approximately 10 mg per dose to approximately 1200 mg per dose. For instance, the dosage of the isovaleramide analog may range from approximately 100 mg per dose to approximately 1000 mg per dose, such as from approximately 200 mg per dose to approximately 800 mg per dose or from approximately 300 mg per dose to approximately 500 mg per dose. Unit solid oral dosage forms may, for example, include about approximately 10 mg to approximately 800 mg of the isovaleramide analog per tablet or capsule, at a dosage ranging from approximately 0.01 mg/kg to approximately 50 mg/kg body weight. Reduced dosage pediatric chewable and liquid oral dosage forms of the pharmaceutical composition may also be prepared and administered, as known in the art. A pharmaceutical composition of the present invention may also be added to foods or beverages in the form of drops (with a dropper from a concentrated preparation of the pharmaceutical composition) for oral administration. In addition, a pharmaceutical composition of the present invention may be formulated into chewing gum to facilitate oral delivery and absorption.

In addition to oral administration, a pharmaceutical composition of the present invention may be administered parenterally, such as by injection. Alternatively, a pharmaceutical composition of the present invention may be prepared for and administered transdermally, transmucosally, intravenously, intraperitoneally, subcutaneously, rectally, nasally, bucally, or intramuscularly.

Therapeutic activity of isovaleramide analogs of the present invention may be determined in an animal model of the CNS condition or disease, as known in the art. For instance, the effect of isovaleramide analogs on spasticity may be determined in a conventional animal model, such as a mutant spastic mouse model, an acute decerebrate rat model, an acute or chronic spinally transected rat model, a chronically spinal cord-lesioned rat model, a Primary Observation Irwin Test in rats, or a Rotarod Test in rats or mice. The effect of the isovaleramide analogs of the present invention on anticonvulsant activity may be determined in a conventional animal model, such as in the Frings audiogenic seizure-susceptible mouse model, which is a model of reflex epilepsy. The effect of the isovaleramide analog of the present invention may also be determined in the Maximal Electroshock (“MES”) seizure test, which is a highly predictive animal seizure model of human generalized tonic-clonic seizures. Drugs that are effective in the MES test are thought to block seizure spread and are likely to be useful for the management of human primary and secondarily generalized tonic-clonic seizures.

The effect of the isovaleramide analogs of the present invention on effective mood disorders may be determined in an amphetamine-induced hyperactivity model in rats, which is a conventional test for classical and atypical antipsychotic activity and for manic behavior. The effect of the isovaleramide analogs of the present invention on migraine headaches may be determined in a conventional animal model of neurogenic inflammation of the meninges. The effect of the isovaleramide analogs of the present invention on neuropathic pain may be measured in a conventional animal model that determines analgesic properties, such as writhing, hotplate, tail flick, arthritic pain, paw pressure tests, and the Bennet or Chung model of neuropathic pain. The effect of the isovaleramide analogs of the present invention on movement disorders and restlessness syndromes may be determined by a conventional animal model, such as the drug-induced akathisias, serotonin syndrome, or rotation induced by unilateral nigral lesions models. The predictive effect of the isovaleramide analogs of the present invention on eliciting neuroprotection and affecting mood disorders and substance abuse/craving may be determined in the kindling seizure animal model or in a stroke animal model. The effect of the isovaleramide analogs of the present invention on anxiety-related disorders may be determined using a conventional anxiolytic animal model, such as the exploratory behavior test or the Vogel Conflict Paradigm. These animal models are described in U.S. Pat. Nos. 5,506,268 and 6,589,994 to Balandrin et al. and to Artman et al., respectively, the disclosure of each of which is incorporated by reference herein in its entirety.

The following examples serve to describe embodiments of the present invention in more detail. These examples are not to be construed as being exhaustive or exclusive as to the scope of this invention.

### EXAMPLES

The synthesis and characterization of many of the isovaleramide analogs shown in FIG. 1 are described below. Starting materials and reagents used in the syntheses were obtained commercially from Sigma-Aldrich Chemical Co., Acros Organics B.V.B.A., Pfaltz & Bauer, or Fluka.

Anticonvulsant activity of some of the isovaleramide analogs shown in FIG. 1 was determined in the Frings Seizure Assay or in the MES test.

### Example 1

### Synthesis of Compound A

Ammonia gas was bubbled through a solution of DL-2-methylbutyryl chloride (10.21 g, 84.7 mmol) in anhydrous tetrahydrofuran (“THF”) (200 mL) for 2 minutes. The DL-2-methylbutyryl chloride was obtained from Acros Organics. The reaction mixture was then capped and stirred for 15 minutes. The precipitated ammonium chloride was then filtered twice through paper. The filtrate was rotary evaporated (20° C.) giving 7.88 g (92.0% yield) of product as a white crystalline solid. This material was dissolved in refluxing ethyl acetate (“EtOAc”) (15 mL). The crystallizing solution was allowed to stand at 20° C. for 30 minutes and then at 0° C. for 5 minutes. The resultant crystals were filtered (no washing). The resultant crystals were allowed to dry to the open air for 14 hours. This yielded 6.73 g (78.6% yield) of Compound A as white crystalline needles.

### Example 2

### Synthesis of Compound B

Under argon, oxalyl chloride (30 mL, 44 g, 340 mmol, 2.0 equiv) was added to a solution of 2,2-dimethylbutyric acid (20.00 g, 172.2 mmol, 1 equiv) in dichloromethane (60 mL) over a period of 1 minute. The reaction solution showed vigorous gas evolution and went from faint yellow to deep red over a period of 3 minutes. The reaction mixture was stirred at 20° C. for 60 minutes (gas evolution ceased sometime between 45-50 minutes). The reaction mixture was then distilled through a short-path distillation apparatus. Dichloromethane and oxalyl chloride were distilled at 39° C.-74° C. The product was distilled at 124° C.-127° C., yielding 15.82 g (68.3% yield) of product, an intermediate acid chloride, as a colorless free-flowing liquid.

Ammonia gas was bubbled through a solution of the intermediate acid chloride (10.24 g, 76.07 mmol) in anhydrous THF (200 mL) for 2 minutes. The reaction mixture was then capped and stirred for 15 minutes. The precipitated ammonium chloride was then filtered twice through paper. The filtrate was rotary evaporated (20° C.), giving 7.67 g (87.5% yield) of product as a white crystalline solid. This material was dissolved in hot EtOAc (20 mL). The crystallizing solution was allowed to stand at 20° C. for 45 minutes and then at 0° C. for 15 minutes. The resultant crystals were filtered (no washing) and dried to the open air for 22 hours. This yielded 6.46 g (73.7% yield) of Compound B as white crystalline plates.

### Example 3

### Synthesis of Compound C

To a solution of Mg(OH)₂ (2.68 g, 45.9 mmol, 1.0 equiv) in water (50 mL) was added isovaleric acid (10 mL, 9.38 g, 91.7 mmol, 2.0 equiv). The reaction mixture was heated to reflux for 18 hours. The cloudy reaction mixture was then hot filtered through paper and chilled in an ice bath. Crystals began to form after chilling for 5 hours. The stoppered solution was scratched with a glass rod and then placed in a refrigerator at +4° C. for approximately 3 days (67 hours). The solution was then evaporated under vacuum (25 mm, 75° C., 30 min) to provide 13.76 g of a white powder with the faint odor of isovaleric acid. This material was dissolved in refluxing 2-propanol (40 mL) and acetonitrile (40 mL) was added slowly at reflux. The heat was removed and the product allowed to crystallize at +4° C. After 4 hours, the volatiles were evaporated and the solid residue was dissolved in water (100 mL) and washed with diethyl ether (3×33 mL). The aqueous layer (pH 8) was evaporated under vacuum (10 mm, 70° C.) and dried for 18 hours at 0.1 mm and 25° C. to provide 6.89 g, 66.4% yield of Compound C as a white powder (ground with mortar/pestle). The white powder had a faint odor of isovaleric acid and a melting point of 156° F.-190° F. (240° C.-244° C. melting).

### Example 4

### Alternate Synthesis of Compound C

To a solution of Mg(OH)₂ (2.68 g, 45.9 mmol, 1.0 equiv) in absolute ethanol (50 mL) was added isovaleric acid (10 mL, 9.38 g, 91.7 mmol, 2.0 equiv). The reaction mixture was heated to reflux for 18 hours. The cloudy reaction mixture was then hot filtered through paper and chilled in an ice bath. No crystals were present after chilling for 5 hours. The stoppered solution was scratched with a glass rod and then placed in a refrigerator at +4° C. for 3 days (67 hours). A few (<20 mg) crystal warts appeared after 3 days. The solution was then evaporated under vacuum (25 mm, 75° C., 30 min) to provide 9.14 g of a shiny, bubbly solid with the faint odor of isovaleric acid. This material was dissolved in refluxing 2-propanol (60 mL) and acetonitrile (10 mL) was added slowly at reflux. The heat was removed and the product allowed to crystallize at +4° C. After 4 hours, the solid was collected on fritted glass and then dried under vacuum for 18 hours at 0.1 mm and 25° C. to provide 4.87 g, 46.9% yield of Compound C as a white powder (ground with mortar/pestle). The white powder had a faint odor of isovaleric acid and a melting point of 180° F.-205° F. (251° C.-259° C. melting).

### Example 5

### Synthesis of Compound D

Ammonia (gas) was bubbled through a solution of 3,3-dimethylacryloyl chloride (5.0 g, 42.17 mmol) in anhydrous THF (100 mL) at 5° C. for 15 minutes. The reaction mixture was stirred overnight at room temperature under static house-nitrogen. The precipitated ammonium chloride, was filtered and washed with THF (100 mL). The filtrate and wash solution were combined and evaporated under reduced pressure. The resulting white solid was redissolved in EtOAc (300 mL). The EtOAc layer was washed with water, 1.0 M HCl, a saturated solution of sodium bicarbonate, and a brine solution. Then, the EtOAc solution was dried over magnesium sulfate, filtered, and evaporated under reduced pressure. The resulting white solid was triturated with a chilled solution of diethyl ether and hexane (50/50 v/v) to afford 0.674 grams of Compound D as white flakes (16% yield). This material was determined to be 100% pure by GC-MS. ¹H NMR gave signals consistent with the Compound D's structure and indicated >98% purity.

### Example 6

### Synthesis of Compound F

Ammonia gas was bubbled through a solution of γ-methylvaleroyl chloride (10.23 g, 76.00 mmol) in anhydrous THF (200 mL) for 3 minutes. The γ-methylvaleroyl chloride was obtained from Pfaltz & Bauer. The reaction mixture was then capped and stirred for 15 minutes. The precipitated ammonium chloride was then filtered twice through paper. The filtrate was rotary evaporated (20° C.) giving 10.42 g (119% yield) of product as a cream-colored crystalline solid. This material was dissolved in refluxing EtOAc (25 mL). The crystallizing solution was allowed to stand at 20° C. for 2.5 hours and then at 0° C. for 15 minutes. The resultant crystals were filtered, washed with cold (0° C.) EtOAc (1×25 mL), and dried to the open air for 64 hours. This yielded 4.95 g (56.5% yield) of Compound F as white crystalline plates.

### Example 7

### Synthesis of Compound J

To a suspension of isovaleramide (10.9 g, 108 mmol, 1 equiv) in diethyl ether (“Et₂O”) (400 mL) was added phosphorus pentasulfide (7.5 g, 17 mmol, 0.16 equiv) in portions over a period of 20 minutes. The isovaleramide was obtained from Lancaster Synthesis. After 2 hours, GC/MS showed 7% starting material. Additional phosphorus pentasulfide (1.5 g, 3.4 mmol, 0.031 equiv) was added. After 10 minutes, GC/MS showed 4% starting material. Additional phosphorus pentasulfide (1.5 g, 3.4 mmol, 0.031 equiv) was added. After 25 minutes, the reaction mixture was filtered through paper, the filtrate was rotary evaporated (50° C.), put under high vacuum (180 mtorr) for 30 minutes, and cooled at −20° C. for 87 hours. This gave 10.25 g (81.2% yield) of a yellow oil. This oil was flash chromatographed (500 mL hexanes, 500 mL 1:1 hex/benzene, 500 mL benzene, 500 mL CHCl₃, 500 mL 1:1 MeOH/CHCl₃) through flash silica gel (100 mm×50 mm diameter). The benzene fraction produced 0.55 g of pure product as a crystalline solid, the CHCl₃ fraction produced 1.89 g of pure product as a crystalline solid, and the 1:1 MeOH/CHCl₃ fraction gave 7.40 g of a slightly impure product as a yellow oil. This yellow oil was flash chromatographed (1000 mL CHCl₃) through flash silica gel (100 mm×50 mm diameter). Fraction #1 gave 2.38 g of slightly impure product as a yellow crystalline solid. Fraction #2 gave 0.68 g of pure product as a crystalline solid. The three pure products were combined and dissolved in Et₂O (30 mL). Petroleum ether (38° C.-56° C.; 60 mL) was added and a large amount of crystalline solid was immediately formed. The crystallizing solution was allowed to stand for 1 hour. The crystals were filtered, washed with petroleum ether (2×20 mL), and dried under high vacuum (150 mtorr) for 17 hours. This yielded 1.56 g (12.4%) of pure Compound J as a white crystalline solid.

### Example 8

### Synthesis of Compound K

Ammonia (gas) was bubbled through a solution of isopropylsulfonyl chloride (10 mL, 12.7 g, 89.1 mmol) in THF (180 mL) at room temperature for 3 minutes. The reaction mixture was capped and stirred for 15 minutes. The precipitated ammonium chloride was then filtered through paper. The solid was washed with THF (25 mL). Additional precipitate was noticed after filtration so the process was repeated. The combined filtrate and washings were evaporated under vacuum (30° C., 10 mm) to provide 10.18 g, 92.8% yield, of a yellow oil. The oil was dissolved in THF (25 mL), poured into diethyl ether (100 mL), and allowed to chill at +4° C. overnight. No crystals were formed so the oil was sublimed on a Kugelrohr apparatus (105° C.-135° C., 0.3 mm) to provide 6.3 g of an orange oil, which crystallized on standing (low melting solid). This material was recrystallized from hot CHCl₃ (15 mL)/ether (10 mL) with seeding to provide Compound K.

### Example 9

### Synthesis of Compound L

Ammonia (gas) was bubbled through a solution of 1-propanesulfonyl chloride (10 mL, 12.7 g, 89.1 mmol) in THF (180 mL) at room temperature for 3 minutes. The reaction mixture was capped and stirred for 15 minutes. The precipitated ammonium chloride was then filtered through paper. The solid was washed with THF (25 mL). Additional precipitate was noticed after filtration so the process was repeated. The combined filtrate and washings were evaporated under vacuum (30° C., 10 mm) to provide 11.03 g, 100% yield, of an orange oil. ¹H NMR of the crude material showed a sulfonamide singlet integrating for 2 protons at 5.5 ppm. The residue was dissolved in diethyl ether (15 mL) and allowed to chill at +4° C. overnight. No crystals were formed so the residue was sublimed on a Kugelrohr apparatus (100° C.-120° C., 0.3 mm) to provide 6.53 g, 59.5% yield, of Compound L as an almost colorless oil, which crystallized on standing (low melting solid). ¹H NMR 5.17 s, 3.12 t, 1.88 q, 1.07 t; GC/MS rt 2.58 min, m/z 124.

### Example 10

### Synthesis of Compound M

Ammonia (gas) was bubbled through a solution of 1-butanesulfonyl chloride (10 mL, 12.1 g, 77.3 mmol) in THF (180 mL) at room temperature for 3 minutes. The reaction mixture was capped and stirred for 15 minutes. The precipitated ammonium chloride was then filtered through paper. The solid was washed with THF (25 mL). Additional precipitate was noticed after filtration so the process was repeated. The combined filtrate and washings were evaporated under vacuum (30° C., 10 mm) to provide 8.70 g, 82.0% yield, of an orange oil. The residue was dissolved in diethyl ether (10 mL) and allowed to chill at +4° C. overnight to provide 3.81 g, 35.9% yield, of Compound M as an almost colorless powder, which was washed with ether/pentane.

### Example 11

### Synthesis of Compound O

Sodium sulfite (98+%, ACS reagent, 200 g) was stirred with water (320 mL) for 1 hour at 25° C. until saturated. To 250 mL of this solution, containing approximately 133 g, 1.06 mol of Na₂SO₃, was added 1-bromo-2-methylpropane (75.6 g, 0.552 mol). The mixture was heated to reflux on a hot plate for 5 days, until the two layers disappeared. The solvent was then removed to provide 223 g of shiny plates. This material was refluxed in 300 mL of 75% ethanol/water, hot filtered to remove the insoluble NaBr, and chilled in an ice bath to yield 32.3 g, 36.5% yield, of Compound O as shiny plates. The filtrate was evaporated to half of its volume and chilled in an ice bath to provide 31.2 g, 37.3% yield, of shiny white plates. The combined weight was 63.4 g and the total yield was 71.7%. The white plates were dried under vacuum overnight and tested for bromide with AgNO₃.

### Example 12

### Synthesis of Compound R

Under argon, oxalyl chloride (35 mL, 51 g, 400 mmol, 2.0 equiv) was added to a solution of cyclopropylacetic acid (20.35 g, 203.3 mmol, 1 equiv) in dichloromethane (70 mL) over a period of 1 minute. The cyclopropylacetic acid was obtained from Lancaster Synthesis. The reaction mixture showed vigorous gas evolution. The reaction mixture was stirred at 20° C. for 2 hours (gas evolution ceased sometime between 1-1.5 hours). The reaction mixture was then distilled through a short-path distillation apparatus. Dichloromethane and oxalyl chloride were distilled at 37° C.-72° C. The product was distilled at 125° C.-126.5° C., yielding 18.97 g (78.72% yield) of product as a colorless free-flowing liquid. The intermediate acid chloride, a chloride of cyclopropylacetic acid, had a molecular formula of C₅H₇ClO, a formula weight of 118.56, and a boiling point of 120° C.-121° C.

Ammonia gas was bubbled through a solution of the intermediate acid chloride (10.03 g, 84.60 mmol) in anhydrous THF (200 mL) for 2 minutes. The reaction mixture was then capped and stirred for 15 minutes. The precipitated ammonium chloride was then filtered twice through paper. The filtrate was rotary evaporated (25° C.), giving 8.37 g (99.8% yield) of product as white crystalline plates. This material was dissolved in refluxing EtOAc (50 mL). The crystallizing solution was allowed to stand at 20° C. for 20 minutes and then at 0° C. for 20 minutes. The resultant crystals were filtered, washed with cold (0° C.) EtOAc (2×25 mL), and dried to the open air for 17 hours. This yielded 5.97 g (71.2% yield) of Compound R as white crystalline plates.

### Example 13

### Synthesis of Compound U

Under argon, oxalyl chloride (35 mL, 51 g, 400 mmol, 2.0 equiv) was added to a solution of 2-methylcyclopropanecarboxylic acid (20.20 g, 201.8 mmol, 1 equiv) in dichloromethane (70 mL) over a period of 1 minute. The 2-methylcyclopropanecarboxylic acid was obtained from Fluka (Buchs, Switzerland). The reaction mixture showed vigorous gas evolution. The reaction mixture was stirred at 20° C. for 75 min (gas evolution ceased sometime between 60-75 min). The reaction mixture was then distilled through a short-path distillation apparatus. Dichloromethane and oxalyl chloride were distilled at 28° C.-63° C. The product was distilled at 126.5° C.-131° C., yielding 20.34 g (85.03% yield) of product, an intermediate acid chloride, as a colorless free-flowing liquid. ¹H NMR showed two diastereomers. Based on the methyl peaks, the product is 12% of one diastereomer and 88% of the other diastereomer.

Ammonia gas was bubbled through a solution of the intermediate acid chloride (10.07 g, 84.94 mmol) in anhydrous THF (200 mL) for 2 minutes. The reaction mixture was then capped and stirred for 10 minutes. The precipitated ammonium chloride was then filtered twice through paper. The filtrate was rotary evaporated (25° C.), giving 7.66 g (91.0% yield) of product as a white crystalline solid. This material was dissolved in refluxing EtOAc (40 mL). The crystallizing solution was allowed to stand at 20° C. for 40 minutes and then at 0° C. for 20 minutes. The resultant crystals were filtered (not washed) and dried to the open air for 16 hours. This yielded 5.00 g (59.4% yield) of Compound U as a white crystalline solid. ¹H NMR showed two diastereomers. The product is about 8% of the lesser diastereomer.

### Example 14

### Synthesis of Compound V

Under argon, oxalyl chloride (25 mL, 36 g, 290 mmol, 2.0 equiv) was added to a suspension of 2-dimethyl-3-dimethyl-cyclopropanecarboxylic acid (20.24 g, 142.3 mmol, 1 equiv) in dichloromethane (50 mL) over a period of 3 minutes. The reaction mixture showed vigorous gas evolution. The reaction mixture was stirred at 20° C. for 45 minutes (gas evolution ceased after 40 minutes). Dichloromethane and oxalyl chloride were distilled at 27° C.-57° C. through a short-path distillation apparatus. The product was sublimed on a Kugelrohr apparatus. Two cuts were taken. The initial cut (18° C., 15 mtorr) gave 4.91 g (21.5% yield) of product, an intermediate acid chloride, as white crystalline needles with a small amount of a free-flowing colorless liquid. The second cut (18° C.-60° C., 15 mtorr) gave 16.13 g (70.5%) of the product as white crystalline needles. ¹H NMR showed the second cut to be much purer than the first cut.

Ammonia gas was bubbled through a solution of the intermediate acid chloride (10.35 g, 64.43 mmol) in anhydrous THF (200 mL) for 2 minutes. The reaction mixture was then capped and stirred for 15 minutes. The precipitated ammonium chloride was then filtered twice through paper. The filtrate was rotary evaporated (20° C.) giving 8.23 g (90.5% yield) of product as a white crystalline solid. This material was dissolved in warm EtOAc (15 mL), and hexanes (60 mL) were added. The crystallizing solution was allowed to stand at 20° C. for 1 hour. The resultant crystals were filtered, washed with hexanes (1×25 mL), and dried under high vacuum (125 mtorr) for 48 hours to yield Compound X as a white, finely crystalline solid.

### Example 15

### Synthesis of Compound W (trans-4-methylcyclohexanecarboxamide)

In a round-bottomed flask, a solution of 4-methylcyclohexanecarboxylic acid (4.78 g, 34 mmol) in thionyl chloride (8 mL) was heated at reflux for 1.5 hours. After this time, a short-path distillation head was attached and the excess thionyl chloride removed. The intermediate acid chloride was dissolved in dichloromethane (10 mL) and added to a stirred solution (−78° C.) of ammonia in diethyl ether (200 mL). The mixture was stirred 2 hours at −78° C. and then allowed to warm to ambient temperature, where it was stirred for 16 hours. The resulting precipitate was collected and washed with diethyl ether. Recrystallization of this material from hot ethyl acetate afforded 2.1 g (44%) of trans-4-methylcyclohexanecarboxamide. GC/MS showed a single component: Rt=6.60 min (100%) m/z (rel. int.) 141 (M+, 39), 126 (21), 112 (13), 98 (32), 86 (19), 72 (55), 59 (39), 55 (100), and 44 (51).

### Example 16

### Synthesis of Compound X (2-(4-methylcyclohexyl)acetamide)

In a round-bottomed flask, a solution of 2-(4-methylcyclohexyl)acetic acid (5 g, 32 mmol) in thionyl chloride (10 mL) was heated at reflux for 4 hours. After this time, a short-path distillation head was attached and the excess thionyl chloride removed. The intermediate acid chloride was dissolved in dichloromethane (10 mL) and added to a stirred solution (−78° C.) of ammonia in diethyl ether (200 mL). The mixture was stirred for 15 minutes at −78° C. and then allowed to warm to ambient temperature, where it was stirred for 16 hours. After this time, the reaction mixture was transferred to a separatory funnel and washed with 1 N HCl (3×50 mL), 1 N NaOH (3×50 mL), and brine (50 mL). The remaining organic solution was dried over anhydrous MgSO₄, filtered, and concentrated to afford 3.3 g (66% yield) of 2-(4-methylcyclohexyl)acetamide. GC/MS showed the material to be composed of two geometric isomers in the ratio of 36:64: Rt=6.60 min (36%) m/z (rel. int.) 155 (M+, 0.3), 140 (0.2), 112 (0.3), 98 (2.4), 95 (2.6), 81(2.4), 69 (2.5), 67 (2.5), 60 (17), and 59 (100); Rt=6.74 min (64%) m/z (rel. int.) 155 (M+, 1.0), 140 (0.5), 112 (0.4), 111 (0.5), 99 (1.5), 98 (5.5), 95 (2.6), 81 (3.1), 69 (2.7), 67 (3.0), 60 (15), and 59 (100).

### Example 17

### Synthesis of Compound HH

A mixture of N-2,2-diisopropylpropanenitrile (5.0 g, 35.9 mmol) in water (2.5 mL) was treated with H₂SO₄ (14.7 g, 150.1 mmol) at 0° C. (ice/brine bath). The reaction suspension was subjected to microwave radiation (Sample absorption: High, Fixed hold time: Yes, Pre-stirring: 20s) at 150° C. for 900 seconds. The reaction solution was transferred into a separatory funnel with EtOAc (70 mL) and water (2.0 mL). The mixture was equilibrated and the aqueous phase removed. The EtOAc layer was washed with 1.0 M NaOH, water, and brine. The EtOAc layer was dried over anhydrous MgSO₄. Excess EtOAc was removed under reduced pressure to afford a crude pale yellow oil. The crude material was purified by Biotage Horizon System (Column Si 40+M 0344-1, 1:1 hexane/diethyl ether), yielding 1.85 grams of an off-white solid (33% yield). This material was determined to be 100% pure by GC/MS. ¹H-NMR gave signals consistent with the product's (Compound HH's) structure and indicated greater than 98% purity.

### Example 18

### Synthesis of Compound II

To 207 mL of 98% H₂SO₄ in a 500 mL 3-neck round bottom flask, which was fitted with a magnetic stirrer bar, a dropping funnel, and a reflux condenser, 72 mL of formic acid was added dropwise at 5° C. over a period of about 15 minutes. A solution of 2,2,3-trimethyl-2-butanol (10.0 g, 1.89 mol) in 50 mL of CCl₄ was then added dropwise to the solution for 5 hours by dropping funnel. The stirring was continued overnight at 5° C. The resulting solution was then quenched with ice (200 g) and the reaction mixture was transferred into a separatory funnel using diethyl ether (500 mL). The reaction mixture was equilibrated and the aqueous phase was extracted two more times with diethyl ether (200 mL). The combined diethyl ether extracts were washed with a 5% aqueous solution of sodium carbonate (2×200 mL). The two alkaline solutions were combined and then acidified with 12 N HCl solutions. The acidic aqueous solution was extracted with diethyl ether (2×300 mL). The extracts were combined and washed with brine and dried over MgSO₄. The excess diethyl ether was evaporated under reduced pressure at room temperature to afford 7.26 grams of a white-colored pasty solid (59% yield). This material was determined to be 95% pure by GC/MS. ¹H NMR gave signals consistent with the intermediate's structure and indicated greater than 98% purity.

The white-colored pasty solid (3.0 g, 24.27 mmol) described above was dissolved in dichloromethane (100 mL) and DMF (0.2 mL) to form a reaction solution, which was treated with oxalyl chloride (2.96 mL, 33.98 mmol) at 0° C. under static in-house nitrogen to form an intermediate acid chloride. The reaction solution was stirred at room temperature overnight under nitrogen. The excess dichloromethane was removed under reduced pressure. Ammonia (gas) was bubbled through a solution of the acid chloride in anhydrous THF (100 mL) at 5° C. for 15 minutes. The reaction mixture was stirred overnight at room temperature under static house-nitrogen. The resulting white precipitate (ammonium chloride) was filtered and washed with THF (50 mL). The filtrate and wash solution were combined and evaporated under reduced pressure.

The resulting white solid was redissolved in diethyl ether (200 mL). The diethyl ether layer was washed with water, 1.0 M HCl, a saturated solution of sodium bicarbonate, and a brine solution. Then, the diethyl ether solution was dried over magnesium sulfate, filtered, and evaporated under reduced pressure to afford a white solid. The resulting white solid was triturated with chilled hexane (50 mL) to afford 2.0 grams of Compound II as a white solid (57.8% yield). This material was determined to be 100% pure by GC/MS. ¹H NMR gave signals consistent with the product's (Compound II's) structure and indicated greater than 98% purity.

### Example 19

### Synthesis of Compound JJ

A solution of ethyl-isobutyrate (10 g, 86 mmol) in dry THF (75 mL) was treated with LDA (48 mL [2.0], 95 mmol) at −78° C. under static house-nitrogen. The reaction mixture was placed in an ice bath and the reaction solution was stirred at 0° C. for 45 minutes. The reaction mixture was placed in an acetone/dry ice bath to maintain the temperature of the reaction mixture between minus 10° C.-15° C. This mixture was treated dropwise with a 2-bromo-propane solution (14 g in 25 mL of THF). The reaction mixture was stirred at room temperature overnight under static nitrogen. The reaction mixture was quenched with a saturated solution of NH₄Cl and transferred into a separatory funnel using brine (200 mL) and diethyl ether (300 mL). The reaction mixture was equilibrated and the aqueous layer removed. The aqueous layer was extracted an additional one time with diethyl ether (300 mL). The combined organic extracts were dried over anhydrous MgSO₄, filtered, and concentrated under reduced pressure to afford 9.67 grams of a yellow-orange liquid (71% yield). The resulting pale-yellow oil was determined to be 86% pure by GC/MS. This crude material was used in the next reaction step (hydrolysis of nitrile to corresponding amide) without further purification.

A crude solution of the pale-yellow oil (25.67 g, 61 mmol) in ethanol (50 mL) was treated with a NaOH solution (4.1 g, 250 mmol, in 50 mL of deionized water). The reaction mixture was stirred at room temperature overnight. The reaction mixture was transferred to a separatory funnel using water (200 mL) and diethyl ether (200 mL). The reaction mixture was equilibrated and the ether layer was removed. The aqueous layer was acidified by a HCl solution (pH˜2) and extracted with diethyl ether (300 mL). The organic layer was dried over magnesium sulfate, filtered, and concentrated under reduced pressure to afford 1.7 grams of a crude intermediate carboxylic acid. This crude material was determined to be 92% pure by GC/MS. ¹H NMR gave signals consistent with the product's structure and indicated greater than 90% purity. This material was used in the next reaction step without further purification.

A crude solution of the intermediate carboxylic acid (1.7 g, 13 mmol) in dichloromethane (100 mL) and DMF (0.1 mL) was treated with oxalyl chloride (1.6 mL, 18.3 mmol) at 0° C. under static in-house nitrogen to afford an intermediate acid chloride. The reaction solution was stirred at room temperature overnight under nitrogen. The excess dichloromethane was removed under reduced pressure. Ammonia (gas) was bubbled through a solution of the acid chloride in anhydrous THF (100 mL) at 5° C. for 15 minutes. The reaction mixture was stirred overnight at room temperature under static house-nitrogen.

The white precipitate (ammonium chloride) was filtered and washed with THF (100 mL). The filtrate and wash solution were combined and evaporated under reduced pressure. The resulting white solid was redissolved in ethyl acetate (300 mL). The ethyl acetate layer was washed with water, 1.0 M HCl, a saturated solution of sodium bicarbonate, and a brine solution. Then, the ethyl acetate solution was dried over magnesium sulfate, filtered, and evaporated under reduced pressure. The resulting white solid was triturated with a chilled solution of diethyl ether and hexane (50/50) to afford 0.537 grams of Compound JJ as a white solid (31.7% yield). This material was determined to be 100% pure by GC/MS. ¹H NMR gave signals consistent with the product's (Compound JJ's) structure and indicated greater than 98% purity.

### Example 20

### Synthesis of Compound KK

A solution of lithium diisopropylamide (“LDA”) (100 mL, 0.2 M) at 0° C. was treated (dropwise) with an isovaleric acid solution (10.80 mL, 0.098 M) in 35 nL of anhydrous THF. The reaction solution was stirred for 30 more minutes upon completion of the addition of the isovaleric acid. The reaction solution, which was a dark-red color, was treated with a solution of 2-iodopropane (29.4 mL, 0.294 M) and hexamethylphosphoramide (“HMPA”) (25.6 mL, 0.15 M) at 0° C. under static nitrogen (pale-yellow color). The reaction solution was stirred for 3 more hours until it warmed up to room temperature. The reaction mixture was quenched with a saturated solution of NH₄Cl and transferred into a separatory funnel using brine (200 mL) and diethyl ether (300 mL). The reaction mixture was equilibrated and the aqueous layer was removed. The aqueous layer was extracted an additional time with diethyl ether (300 mL). The combined organic extracts were dried over anhydrous MgSO₄, filtered, and concentrated under reduced pressure to afford a yellow-orange liquid, which solidified upon standing at room temperature. This crude material was triturated with 50 mL of hexane to afford 2.9 grams of an off-white solid (21% yield). This material was determined to be 100% pure by GC/MS. ¹H NMR gave signals consistent with the product's structure and indicated greater than 98% purity.

The off-white solid (3.65 g, 25.3 mmol) described above was dissolved in dichloromethane (75 mL) and DMF (0.3 mL) and was treated with oxalyl chloride (2.5 mL, 28 mmol) at 0° C. under static in-house nitrogen to afford an intermediate acid chloride of the off-white solid. The reaction solution was stirred at room temperature overnight under nitrogen. The excess oxalyl chloride was removed under reduced pressure. Ammonia (gas) was bubbled through a solution of the acid chloride in anhydrous dichloromethane (100 mL) at 5° C. for 15 minutes. The reaction mixture was stirred overnight at room temperature under static house-nitrogen.

The white precipitate (ammonium chloride) was filtered and washed with dichloromethane (100 mL). The filtrate and wash solution were combined and washed with water, 1.0 M HCl, a saturated solution of sodium bicarbonate and, a brine solution and were dried over magnesium sulfate, filtered, and evaporated under reduced pressure. The resulting white solid was triturated with a chilled solution of diethyl ether and hexane (50/50) to afford 970 mg of Compound KK as white flakes (34% yield). This material was determined to be 100% pure by GC/MS. ¹H NMR gave signals consistent with the product's (Compound KK's) structure and indicated greater than 98% purity.

### Example 21

### Synthesis of Compound LL

A solution of (1R, 2R)-(−)-pseudoephedrine (32.3 g, 0.195 mol) and triethylamine (36.0 nL, 0.254 mol) in THF (250 mL) was treated with tert-butylacetyl chloride (30.0 μL, 0.215 mol) at 0° C. under house nitrogen. The reaction mixture was stirred at room temperature overnight under static nitrogen. The reaction mixture was transferred to a 1-L separatory funnel using water (100 mL) and diethyl ether (300 mL). The reaction mixture was equilibrated and the organic layer was separated. The diethyl ether layer was washed with 1.0M HCl (60 mL), water (2×100 mL), NaOH (60 mL), water (2×100 mL), and brine (100 mL). The diethyl ether was dried over MgSO₄ and filtered. The excess diethyl ether was removed under reduced pressure, which afforded a pale-yellow slurry. The slurry was triturated with chilled-hexane and filtered to afford 36.7 g of a white solid (71%). This material determined to be pure by GC/MS. ¹H NMR gave signals consistent with the intermediate's structure and indicated greater than 98% purity.

The white solid (10 g, 38 mmol) described above was dissolved in dry THF (100 mL) and was treated dropwise with LDA (7.3 g, 228 mmol, 2.0 M (34 mL)) at −78° C. under static house-nitrogen. The reaction solution was placed in an ice/brine bath for 60 minutes and stirring was continued at 0° C. Then, the reaction solution was treated with iodomethane (5.93 g, 42 mmol) added dropwise into the reaction solution at 0° C. The reaction solution was stirred and allowed to warm up to room temperature under static nitrogen. The reaction mixture was quenched by the addition of a saturated solution of NH₄Cl. The mixture was then transferred into a separatory funnel using diethyl ether (500 mL). The mixture was equilibrated and the diethyl ether layer was removed. The aqueous layer was extracted two additional times with diethyl ether (300 mL). The combined diethyl ether extracts were washed with brine (500 mL), dried over anhydrous MgSO₄, filtered, and concentrated under reduced pressure at room temperature to afford 10.5 g of a crude yellow-orange oil. This crude material was triturated with cold hexane, which afforded 8.34 g of a white powder (79.3% yield). This material was determined to be 100% pure by GC/MS. ¹H NMR gave signals consistent with the intermediate's structure and indicated greater than 98% purity.

The white powder (8.34 g, 30 mmol) described above was dissolved in 80 ml of 1,4-dioxane and was treated with 9.0 M H₂SO₄ solution (50 mL of concentrated H₂SO₄ diluted with 50 mL of water). The reaction mixture was refluxed for 3 hours and then was allowed to cool down to room temperature and quenched with crushed ice (200 g). Then, the reaction mixture was treated with a 12 M NaOH solution and the reaction mixture was adjusted to a pH of 9. The reaction mixture was extracted with dichloromethane (2×300 mL). Then, the aqueous layer was acidified with 18 M HCl and the solution was adjusted to a pH of 2. The acidic aqueous layer was extracted two additional times with dichloromethane (300 mL). The dichloromethane extracts from the acidic aqueous solution were combined and washed with brine and dried over MgSO₄. The excess dichloromethane was removed under reduced pressure at 30° C. This afforded 1.54 g of a pale yellow oil (39% yield). This material was determined to be 100% pure by GC/MS. ¹H NMR gave signals consistent with the intermediate's structure and indicated greater than 95% purity. The optical rotation of the intermediate was calculated as follows:
[α]_{D}²⁴=(100×α)/(1×c)=(100×(−0.831))/(1×15.35)=−5.41±0.01(c4, CH₂Cl₂)
c=1.535 g/10 mL=15.35 g/100 mL, α=observed value from polarimeter.

The pale yellow oil (1.54 g, 12 mmol) described above was dissolved in dichloromethane (50 mL) and DMF (0.11 mL) and was treated with oxalyl chloride (1.4 mL, 15 mmol) at 0° C. under static in-house nitrogen to afford an acid chloride of the pale yellow oil. The reaction solution was stirred at room temperature overnight under nitrogen. The excess dichloromethane was removed under reduced pressure. Ammonia (gas) was bubbled through a solution of the acid chloride in anhydrous THF (100 mL) at 5° C. for 15 minutes. The reaction mixture was stirred overnight at room temperature under static house-nitrogen.

The white precipitate (ammonium chloride) was filtered and washed with THF (50 mL). The filtrate and wash solution were combined and evaporated under reduced pressure. The resulting white solid was redissolved in diethyl ether (200 mL). The diethyl ether layer was washed with water, 1.0 M HCl, a saturated solution of sodium bicarbonate, and a brine solution. Then, the diethyl ether solution was dried over magnesium sulfate, filtered, and evaporated under reduced pressure. The resulting white solid was triturated with chilled hexane (50 mL), affording 0.164 grams of Compound LL as a white solid (11% yield). This material was determined to be 100% by GC/MS. ¹H NMR gave signals consistent with the product's (Compound LL's) structure and indicated greater than 98% purity.

The product was also evaluated by chiral HPLC. The product was dissolved in 5% ethanol in hexane to a concentration of 5 mg/mL. 20 uL of each sample was injected onto a ChiralPak AS-H column (5 μm, 25×0.46 mm i.d.) using 6% ethanol in hexane at 1 ml/min, measuring UV absorbance at 220 nm (and 230 nm, channel 2). The enantiomeric excess was calculated as follows: 95.56−4.44=91.12 e.e (R enantiomer).

### Example 22

### Synthesis of Compound MM

A solution of (1S, 2S)-(+)-pseudoephedrine (32.3 g, 0.195 mol) and triethylamine (36.0 mL, 0.254 mol) in THF (250 mL) was treated with tert-butylacetyl chloride (30.0 mL, 0.215 mol) at 0° C. under house nitrogen. The reaction mixture was stirred at room temperature overnight under static nitrogen. The reaction mixture was transferred to a 1-L separatory funnel using water (100 mL) and diethyl ether (300 mL). The reaction mixture was equilibrated and the organic layer was separated. The diethyl ether layer was washed with 1.0M HCl (60 mL), water (2×100 mL), NaOH (60 mL), water (2×100 mL), and brine (100 mL). The diethyl ether was dried over MgSO₄ and filtered. The excess diethyl ether was removed under reduced pressure, which afforded a pale-yellow slurry. The slurry was triturated with chilled hexane. The mixture was filtered and afforded 35.60 grams of a white solid (69%). This material was determined to be pure by GC/MS. ¹H-NMR signals were consistent with the product's structure and indicated greater than 98% purity.

The white solid (10 g, 38 mmol) described above was dissolved in dry THF (100 mL) and was treated dropwise with LDA (7.3 g, 228 mmol, 2.0 M (34 mL)) at −78° C. under static house-nitrogen. The reaction solution was placed in an ice/brine bath for 60 minutes and stirring was continued at 0° C. The reaction solution was treated with iodomethane (5.93 g, 42 mmol) added dropwise into the reaction solution at 0° C. The reaction solution was stirred and allowed to warm up to room temperature under static nitrogen. The reaction mixture was quenched by the addition of a saturated solution of NH₄Cl. Then, the mixture was transferred into a separatory funnel using diethyl ether (500 mL). The mixture was equilibrated and the diethyl ether layer was removed. The aqueous layer was extracted two additional times with diethyl ether (300 mL). The combined diethyl ether extracts were washed with brine (500 mL), dried over anhydrous MgSO₄, filtered, and concentrated under reduced pressure at room temperature to afford 10.5 grams of a crude yellow-orange oil. This crude material was triturated with cold hexane, which afforded 8.15 g of a white powder (77% yield). This material was determined to be 100% pure by GC/MS. ¹H NMR gave signals consistent with the product's structure and indicated greater than 98% purity.

The white powder (8.15 g, 29.4 mmol) described above was dissolved in 80 ml of 1,4-dioxane and was treated with 9.0 M H₂SO₄ solution (50 mL of concentrated H₂SO₄ diluted with 50 mL of water). The reaction mixture was refluxed for 3 hours. The reaction mixture was allowed to cool down to room temperature and quenched with crushed ice (200 g). The reaction mixture was treated with a 12 M NaOH solution and the pH was adjusted to 9. The reaction mixture was extracted with dichloromethane (2×300 mL). Then, the aqueous layer was acidified with 18 M HCl and was adjusted to a pH of 2. The acidic aqueous layer was extracted two additional times with dichloromethane (300 mL). The dichloromethane extracts from the acidic aqueous solution were combined and washed with brine and dried over MgSO₄. The excess dichloromethane was removed under reduced pressure at 30° C. to afford 1.2 g of a pale yellow oil (31.4% yield). This material was determined to be 98.8% pure by GC/MS. ¹H NMR gave signals consistent with the compound's structure and indicated >98% purity. The optical rotation of the compound was calculated as follows:
[α]_{D}²⁴=(100×α)/(1×c)=(100×2.56)/(1×12)=21.33±0.01(c4, Et₂O)
c=1.2 g/110 mL=12 g/100 mL, α=observed value from polarimeter.

The pale yellow oil (1.2 g, 9.2 mmol) described above was dissolved in dichloromethane (50 mL) and DMF (0.15 mL) and was treated with oxalyl chloride (1.1 mL, 12 mmol) at 0° C. under static in-house nitrogen to afford an intermediate acid chloride of the pale yellow oil. The reaction solution was stirred at room temperature overnight under nitrogen. The excess dichloromethane was removed under reduced pressure. Ammonia (gas) was bubbled through a solution of the acid chloride in anhydrous THF (100 mL) at 5° C. for 15 minutes. The reaction mixture was stirred overnight at room temperature under static house-nitrogen.

The white precipitate (ammonium chloride) was filtered and washed with THF (50 mL). The filtrate and wash solution were combined and evaporated under reduced pressure. The resulting white solid was redissolved in diethyl ether (200 mL). The diethyl ether layer was washed with water, 1.0 M HCl, a saturated solution of sodium bicarbonate, and a brine solution. Then, the ether solution was dried over magnesium sulfate, filtered, and evaporated under reduced pressure. The resulting white solid was triturated with chilled hexane (50 mL) to afford 0.463 g of Compound MM as a white solid (39% yield). This material was determined to be 100% pure by GC/MS. ¹H NMR gave signals consistent with the product's (Compound MM's) structure and indicated greater than 98% purity.

The product was also evaluated by chiral HPLC. The product was dissolved in 5% ethanol in hexane to a concentration of 5 mg/mL. 20 uL of each sample was injected onto a ChiralPak AS-H column (5 μm, 25×0.46 mm i.d.) using 6% ethanol in hexane at 1 ml/min, measuring UV absorbance at 220 nm (and 230 nm, channel 2). The enantiomeric excess was calculated as follows: 96.62−3.38=93.24 e.e (S enantiomer).

### Example 23

### Anticonvulsant Activity of Compound S in the Frings Seizure Assay

The anticonvulsant activity of compound S ((S)-(+)-2,2-dimethylcyclopropane-carboxamide) was tested using the Frings audiogenic seizure-susceptible mouse. The results in Tables 1-3 demonstrate the anticonvulsant activity of Compound S when administered orally in this animal model of epilepsy. Compound S, which is a solution at 300 mg/kg and a suspension at 600 mg/kg, was administered to the mice at the doses indicated in Tables 1-3. Compound S was obtained from Sigma-Aldrich Chemical Co. (Aldrich catalog number: 43,463-9).

TABLE 1
Effect of Compound S on the Audiogenic Seizure Susceptibility
of Frings Mice Following Oral (p.o.) Administration.

| | | |
|---|---|---|
| Dose | #Protected/#Tested | #Toxic/#Tested |

| (mg/kg) | 30 min | 120 min | 30 min | 120 min |
|---|---|---|---|---|
| 300 | 4/4 | 4/4 | 0/4 | 0/4 |
| 100 | 3/4 | 1/4 | 0/4 | 0/4 |
| 30 | 0/4 | 0/4 | 0/4 | 0/4 |

TABLE 2
Time effect of Compound S Against Audiogenic Seizure Susceptibility
of Frings Mice Following Oral (p.o.) Administration.

| | | Time (hrs) | | | | |
|---|---|---|---|---|---|---|
| | Dose | ¼ | ½ | 1 | 2 | 4 |
| # Prot./# Tested | 100 mg/kg | ¾ | 3/4 | 0/4 | 1/4 | |
| | 75 mg/kg | ¾ | 0/4 | | | |

TABLE 3
Effect of Compound S on the Audiogenic Seizure Susceptibility
of Frings Mice Following Oral (p.o.) Administration.

| | Seizure | | | |
|---|---|---|---|---|
| Dose | Score ± | #Prot./#Tested | ED₅₀ | #Toxic/#Tested |
| (mg/kg) | S.E.M. | at 15 min | (mg/kg) | at 15 min |
| 50 | 5 ± 0 | 0/8 | | |
| 60 | 2.1 ± 0.9 | 5/8 | | |
| 75 | 1.1 ± 0.6 | 7/8 | 64.87 | |
| 100 | 1.6 ± 0.8 | 6/8 | (50.16-78.92)^{a} | |
| 200 | 0.1 ± 0.1 | 8/8 | | |
| 600 | | | | 1/8 |

^{a}95% confidence interval

At the time of testing, individual mice were placed into a round Plexiglas chamber and exposed to a sound stimulus of 110 decibels, 11 kHz, for 20 seconds. Mice not displaying tonic hindlimb extensions upon exposure to the sound stimulus were considered protected. In addition, the seizure score for each mouse was recorded as: (1) running for less than 10 seconds; (2) running for greater than 10 seconds; (3) clonic activity of limbs and/or vibrissae; (4) forelimb extension/hindlimb flexion; and (5) hindlimb extension. The average seizure score was calculated for each group of mice used in the dose-response study. The number of protected mice versus the number of tested mice is shown in Tables 1-3. At a dose of 300 mg/kg, all of the tested mice were protected, as shown in Table 1. At a lower dose of 100 mg/kg, 75% of the mice were protected at 30 minutes after administering Compound S and 25% were protected at 120 minutes after administering Compound S. At 30 mg/kg, no mice were protected. As shown in Table 2, 75% of the mice were protected at 15 and 30 minutes after administering 100 mg/kg of Compound S. At 1 hour after administration, no mice were protected and at 2 hours, 1 mouse was protected. At a dose of 75 mg/kg, 75% of the mice were protected at 15 minutes and no mice were protected at 30 minutes. As shown in Table 3, none of the mice were protected at 15 minutes after administering 50 mg/kg Compound S. At 60 mg/kg of Compound S, 62.5% of the mice were protected at 15 minutes; at 75 mg/kg of Compound S, 87.5% of the mice were protected at 15 minutes; at 100 mg/kg of Compound S, 75% of the mice were protected at 15 minutes; and at 200 mg/kg of Compound S, all the mice were protected at 15 minutes. As shown in Table 3, the average seizure score generally decreased with increasing doses.

The median effective dose (“ED₅₀”) for protection against tonic extension was 64.87 mg/kg (the 95% confidence interval ranged from 50.16 mg/kg to 78.92 mg/kg).

At each dose, the mice were also tested on a rotarod for testing of motor impairment (toxicity). Testing for motor impairment on the rotarod involved placing a mouse for a three-minute trial period on a one-inch diameter rod rotating at six revolutions per minute. If the mouse fell off of the rotating rod three times within the three-minute time period, the dose was considered a toxic response. As shown in Table 1, none of the doses produced toxic responses at 30 minutes or at 120 minutes. As shown in Table 3, 12.5% (1 out of 8) of the mice tested at a dose of 600 mg/kg of Compound S had a toxic response at 15 minutes.

Isovaleramide was also tested in the Frings audiogenic seizure-susceptible mouse model. A comparison of the ED₅₀'s of Compound S and isovaleramide is shown in Table 4.

TABLE 4
Effect of Compound S and Isovaleramide on the
Audiogenic Seizure Susceptibility of Frings
Mice Following Oral (p.o.) Administration.

| Compound | ED₅₀ (mg/kg) | TD₅₀ (mg/kg) |
|---|---|---|
| Compound S | 64.8 | >600 |
| Isovaleramide | 206 | |

Compound S had a 10-fold separation between efficacy and CNS toxicity, as measured by rotarod impairment. In comparison, isovaleramide had a 5-fold separation between efficacy and CNS toxicity, as measured by rotarod impairment

In summary, Compound S demonstrated activity as an anticonvulsant in the Frings audiogenic seizure-susceptible mouse model of reflex epilepsy. The anticonvulsant activity was observed as early as 15 minutes and up to 2 hours after oral administration of Compound S. In addition, Compound S had low toxicity. As such, Compound S exhibited a good separation between activity and toxicity.

### Example 24

### Anticonvulsant Activity of Compounds I, W, X, and AA in the Frings Seizure Assay

The anticonvulsant activity of compounds I, W, X, and AA was tested using the Frings audiogenic seizure-susceptible mouse model as described in Example 23. As shown in Table 5, a racemic mixture and two enantiomers of compound AA were tested. The results in Table 5 demonstrate the anticonvulsant activity of Compounds I, W, X, and AA when administered orally in this animal model of epilepsy. The therapeutic index relates to separation between therapeutic efficacy and CNS toxicity as measured by motor impairment in the rotarod performance test.

TABLE 5
Effect of Compounds I, W, X, and AA In the Audiogenic Seizure
Susceptibility of Frings Mice Following Oral (p.o.) Administration.

| | | ED₅₀ (mg/kg) | |
|---|---|---|---|
| | | Oral | Therapeutic |
| Compound | Structure | Administration | Index |
| I | 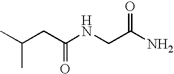 | <1000 | |
| X (cis and trans mixture) | 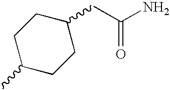 | 86.4 | >6-fold |
| W | 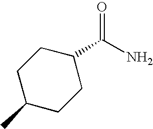 | 164.4 | >4-fold |
| AA (cis and trans mixture) | 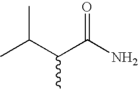 | 140 | |
| AA (S- enantiomer) | 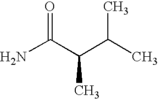 | 84.13 | >4-fold |
| AA (R- enantiomer) | 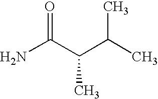 | 84.15 | >4-fold |

The therapeutic index relates to separation between therapeutic efficacy and CNS toxicity as measured by motor impairment in the rotarod performance test.

### Example 25

### Anticonvulsant Activity of Compounds A-H, J-R, T-V, Y, Z, and BB-NN, or Mixtures of Compounds A-NN in the Frings Seizure Assay

The activity of each of compounds A-H, J-R, T-V, Y, Z, and BB-NN, or mixtures of Compounds A-NN are tested in the Frings audiogenic seizure-susceptible mouse model to determine its anticonvulsant activity. Each of the compounds or mixture of compounds is administered orally to the mice, as described in Examples 23 and 24.

Each of the compounds or the mixture of compounds will exhibit activity as an anticonvulsant in the Frings audiogenic seizure-susceptible mouse model. In addition, each of the tested compounds will possess low toxicity. Each of compounds A-H, J-R, T-V, Y, Z, and BB-NN, or mixtures of Compounds A-NN will exhibit a good separation between activity and toxicity.

Each of compounds A-H, J-R, T-V, Y, Z, and BB-NN, or mixtures of Compounds A-NN will possess a similar or increased activity compared to that of isovaleramide. The tested compounds or mixtures of compounds will also demonstrate increased stability and increased half-life compared to isovaleramide.

### Example 26

### Anticonvulsant Activity of Compound S, Compound W, the S-enantiomer of Compound AA, the R-enantiomer of Compound AA, Compound HH, Compound JJ, and Compound MM in the Mouse MES Test

In the MES test, a substance is administered orally (p.o.) and is tested for efficacy against maximal electroshock (MES)-induced tonic extension seizures. Adult male Swiss-Webster mice (each 20-25 g of body weight) were used. The mice were housed in Association for the Assessment and Accreditation of Laboratory Animal Care-approved facilities under a constant 12-hour light/dark cycle with a constant temperature of 21° C.-23° C. and a relative humidity of 30% to 50%. All animals were permitted free access to standard laboratory chow (Prolab RMH 3000; PMI Nutrition International, LLC, Brentwood, Mo.) and water, except when removed from their home cages for testing. All mice were handled in a manner consistent with the recommendations in the National Research Council Publication Guide for the Care and Use of Laboratory Animals.

Test compounds (Compound S, Compound W, the S-enantiomer of Compound AA, the R-enantiomer of Compound AA, Compound HH, Compound JJ, and Compound MM) were dissolved in saline containing 0.5% methylcellulose. To determine time to peak effect, 300 mg/kg of each of the test compounds was suspended in 0.5% methylcellulose in saline and administered orally to a group of 20 mice. For comparative purposes, isovaleramide was also tested. At various times (0.25, 0.5, 1, 2, and 4 hours) after p.o. administration, individual mice were placed on the rotarod and tested for their ability to maintain balance on a rotating (6 rpm) 2.5-cm diameter knurled rod for 60 seconds. Mice that fell three times in this 60-second trial were considered impaired.

Following rotarod testing, the mice were tested in the maximal electroshock (MES)-induced tonic extension seizures test. For this test, a drop of electrolyte solution (0.5% butacaine sulfate in 0.9% saline) was placed on the eyes of each mouse prior to placement of corneal electrodes. The mice were restrained by gripping the loose skin on their dorsal surface and saline-coated corneal electrodes were held lightly against the two corneas. Each mouse received an electrical stimulation (50 mAmp, 50 Hz current, 0.2 sec) delivered through a silver-coated corneal electrode using an electroshock machine as originally described by Woodbury and Davenport (Arch Int. Pharmacodyn. Ther., 92:97-104, 1952). The mice were observed for a period ofup to 30 seconds for the occurrence of a tonic hindlimb extensor response. A tonic seizure was defined as a hindlimb extension in excess of 90 degrees from the plane of the body. Results were treated in a quantal manner. Mice that did not display tonic hindlimb extension after administration of the test compound were considered protected and this was taken as the efficacy endpoint for this test. The results were expressed as # protected (P)/# tested (T) and # impaired (1)/# tested, as shown in Tables 6-10. ED50's (the effective dose at which 50% of the mice were protected) were determined for Compounds W and HH by administering various doses (30, 100, 300 mg/kg) at the previously determined time to peak effect, as shown in Table 11.

TABLE 6
Effect of Compound S in the MES Mouse Assay.

| | | Time of Test (Hours) | | | | |
|---|---|---|---|---|---|---|
| Compound S | Dose (mg/kg) p.o. | 0.25 | 0.5 | 1 | 2 | 4 |
| # P/# T | 300 | 0/8 | 0/8 | 1/8 | 0/4 | 0/4 |
| # I/# T | | 0/8 | 1/8 | 1/8 | 0/4 | 0/4 |

TABLE 7
Effect of the S-enantiomer of Compound
AA in the MES Mouse Assay.

| S-enantiomer | | Time of Test (Hours) | | | | |
|---|---|---|---|---|---|---|
| of Compound AA | Dose (mg/kg) p.o. | 0.25 | 0.5 | 1 | 2 | 4 |
| # P/# T | 300 | 3/4 | 0/4 | 1/4 | 1/4 | 0/4 |
| # I/# T | | 0/4 | 1/4 | 0/4 | 0/4 | 0/4 |

TABLE 8
Effect of the R-enantiomer of Compound
AA in the MES Mouse Assay.

| R-enantiomer | | Time of Test (Hours) | | | | |
|---|---|---|---|---|---|---|
| of Compound AA | Dose (mg/kg) p.o. | 0.25 | 0.5 | 1 | 2 | 4 |
| # P/# T | 300 | 0/4 | 0/4 | 0/4 | 0/4 | 0/4 |
| # I/# T | | 3/4 | 3/4 | 0/4 | 3/4 | |

TABLE 9
Effect of Compound JJ in the MES Mouse Assay.

| | | Time of Test (Hours) | | | | |
|---|---|---|---|---|---|---|
| Compound JJ | Dose (mg/kg) p.o. | 0.25 | 0.5 | 1 | 2 | 4 |
| # P/# T | 300 | 1/4 | 0/4 | 0/4 | 0/4 | 0/4 |
| # I/# T | | 1/4 | 2/4 | 1/4 | 0/4 | 0/4 |

TABLE 10
Effect of Compound MM in the MES Mouse Assay.

| | | Time of Test (Hours) | | | | |
|---|---|---|---|---|---|---|
| Compound MM | Dose (mg/kg) p.o. | 0.25 | 0.5 | 1 | 2 | 4 |
| # P/# T | 300 | 0/4 | 1/4 | 2/4 | 3/4 | 3/4 |
| # I/# T | | 2/4 | 3/4 | 4/4 | 3/4 | 3/4 |

TABLE 11
ED₅₀ of Compounds W and HH in the MES Mouse Assay.

| | ED₅₀ (mg/kg) |
|---|---|
| Compound | Oral Administration |
| W | Inactive at 300 mg/kg |
| HH | 114.1 |
| Isovaleramide | 913 |

### Example 27

### Anticonvulsant Activity of Compounds A-R, T-V, X-Z, BB-GG, II, KK, LL, NN, or Mixtures of Compounds A-NN

The activity of each of compounds A-R, T-V, X-Z, BB-GG, II, KK, LL, NN, or mixtures of Compounds A-NN is tested in the MES test to determine its anticonvulsant activity. Each of the compounds or mixture of compounds is administered orally to the mice, as described in Example 26.

Each of the compounds or the mixture of compounds will exhibit activity as an anticonvulsant in the MES test. In addition, each of the tested compounds will possess low toxicity. Each of compounds A-R, T-V, X-Z, BB-GG, II, KK, LL, NN, or mixtures of Compounds A-NN will exhibit a good separation between activity and toxicity.

Each of compounds A-R, T-V, X-Z, BB-GG, II, KK, LL, NN, or mixtures of Compounds A-NN will possess a similar or increased activity compared to that of isovaleramide. The tested compounds or mixtures of compounds will also demonstrate increased stability and increased half-life compared to isovaleramide.

### Example 28

### CNS Activity of Compounds A-NN or Mixtures Thereof

Each of Compounds A-NN or mixtures of Compounds A-NN is tested in an animal model of spasticity, affective mood disorders, neuropathic pain syndromes, neurodegenerative disorders, headaches, premenstrual syndrome, menstrual discomfort, hyperexcitability in children, restlessness syndromes, movement disorders, cerebral trauma, anxiety-related disorders, or symptoms of substance abuse/craving. An appropriate animal model is selected depending on the CNS condition or disease in which the compound or mixture of compounds is to be tested, as known in the art.

Each of the compounds or mixture of compounds is administered to the animals orally, transdermally, transmucosally, intravenously, intraperitoneally, subcutaneously, rectally, nasally, bucally, or intramuscularly.

Each of the compounds or mixtures of compounds will exhibit CNS activity in the selected animal model. In addition, each of the compounds and mixtures of compounds will possess low toxicity. As such, each of the tested compounds and mixtures of compounds will exhibit a good separation between activity and toxicity. The tested compounds or mixtures of compounds will demonstrate a similar or increased CNS activity compared to that of isovaleramide.

The compounds or mixtures of compounds will also have increased stability and increased half-life compared to isovaleramide.

While the invention may be susceptible to various modifications and alternative forms, specific embodiments have been shown by way of example in the drawing and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

What is claimed is:

1. An isovaleramide analog comprising a compound with at least one of an increased potency, an increased half-life, and an increased stability compared to isovaleramide, wherein the compound is a cyclic analog of isovaleramide or a noncyclic analog of isovaleramide.

2. The isovaleramide analog of claim 1, wherein the compound is selected from the group consisting of 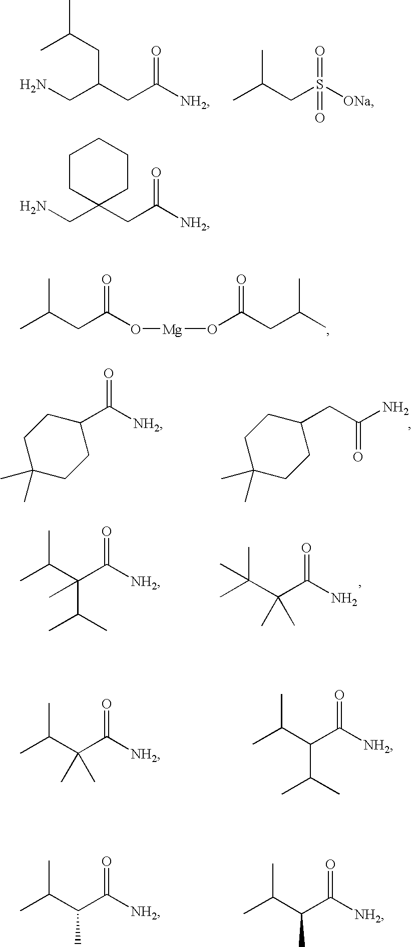
and mixtures thereof.

3. A pharmaceutical composition for treating a central nervous system condition or disease, comprising:
an isovaleramide analog selected from the group consisting of 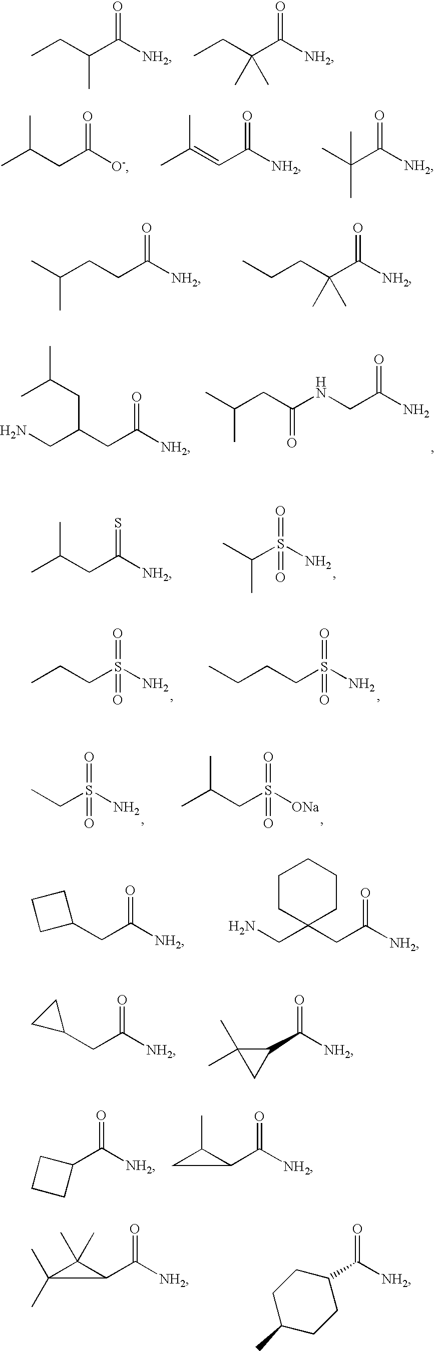 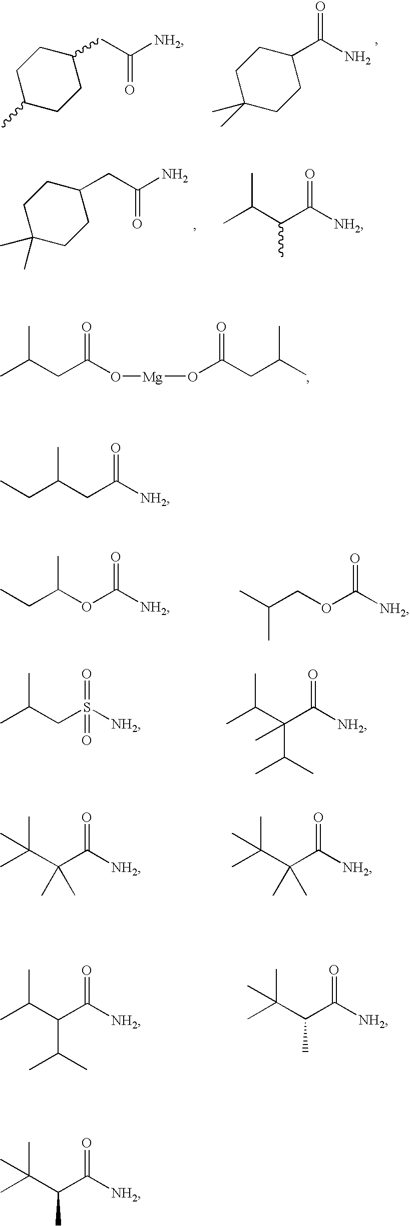
and mixtures thereof; and a pharmaceutically acceptable carrier.

4. The pharmaceutical composition of claim 3, wherein the pharmaceutically acceptable carrier is selected from the group consisting of calcium carbonate, calcium phosphate, calcium sulfate, sucrose, dextrose, lactose, fructose, xylitol, sorbitol, starch, starch paste, cellulose derivatives, gelatin, polyvinylpyrrolidone, sodium chloride, dextrins, stearic acid, magnesium stearate, calcium stearate, vegetable oils, polyethylene glycol, sterile phosphate-buffered saline, saline, Ringer's solutions, and mixtures thereof.

5. The pharmaceutical composition of claim 3, wherein the isovaleramide analog comprises from approximately 1% by weight to approximately 95% by weight of a total weight of the pharmaceutical composition.

6. The pharmaceutical composition of claim 3, wherein the isovaleramide analog is present in the pharmaceutical composition in a range of from approximately 10 mg to approximately 1200 mg.

7. A method of treating a central nervous system condition or disease, comprising:
administering a compound selected from the group consisting of 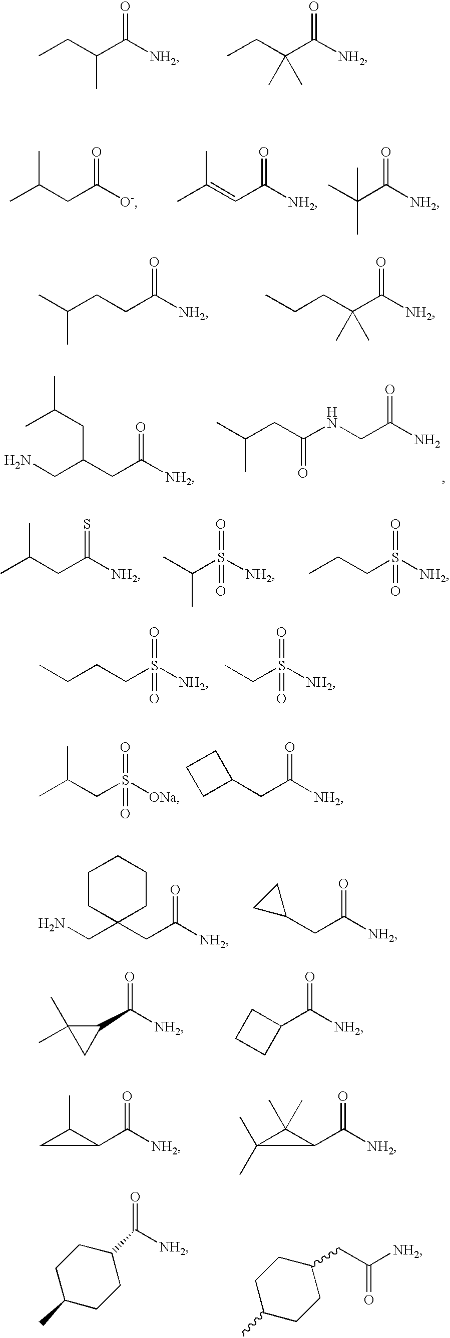 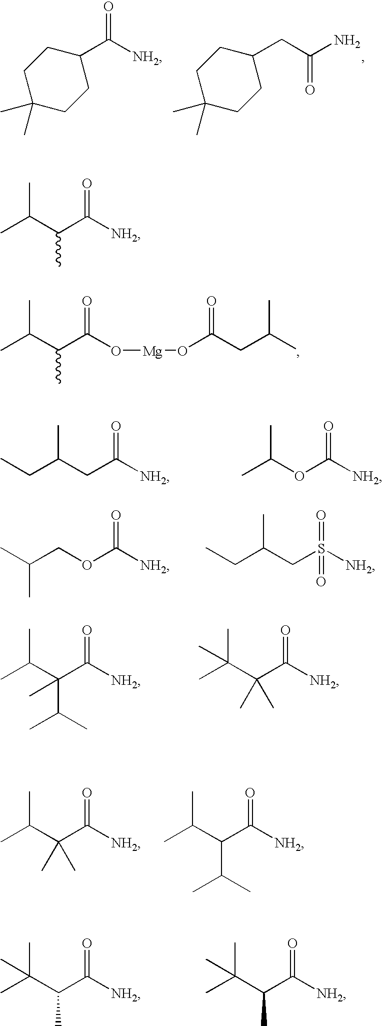
and mixtures thereof to a patient suffering from a central nervous system condition or disease.

8. The method of claim 7, wherein administering the compound comprises administering the compound as an oral dosage form selected from the group consisting of an enteric-coated tablet, a caplet, a gel cap, and a capsule.

9. The method of claim 7, wherein administering the compound comprises administering the compound as a liquid dosage form selected from the group consisting of a syrup and an elixir.

10. The method of claim 7, wherein administering the compound comprises administering the compound orally, transdermally, transmucosally, intravenously, intraperitoneally, subcutaneously, rectally, nasally, bucally, or intramuscularly.

11. The method of claim 7, wherein administering the compound to the patient suffering from the central nervous system condition or disease comprises administering a therapeutically effective amount of the compound to the patient.

12. The method of claim 7, wherein administering the compound to the patient suffering from the central nervous system condition or disease comprises administering from approximately 10 mg to approximately 1200 mg of the compound to the patient.

13. The method of claim 7, wherein administering the compound comprises administering a pharmaceutical composition that comprises the compound and a pharmaceutically acceptable carrier.

14. The method of claim 7, wherein administering the compound to the patient suffering from the central nervous system condition or disease comprises administering the compound to the patient suffering from a central nervous system condition or disease selected from the group consisting of convulsions, spasticity, affective mood disorders, neuropathic pain syndromes, neurodegenerative disorders, headaches, premenstrual syndrome, menstrual discomfort, hyperexcitability in children, restlessness syndromes, movement disorders, cerebral trauma, anxiety-related disorders, and symptoms of substance abuse/craving.
